(19) United States
(12) Patent Application Publication
(10) Pub. No.: US 2004/0013622 A1
(43) Pub. Date: Jan. 22, 2004

(54) Cosmetic compositions for improving the health of nails

(75) Inventor: Ginette Godbout, Ville St-Laurent (CA)

Correspondence Address: OBLON, SPIVAK, MCCLELLAND, MAIER & NEUSTADT, P.C., 1940 DUKE STREET, ALEXANDRIA, VA, 22314, US

(73) Assignee: FORMULE DIAMANCEL, INC., Ville Saint-Laurent (CA)

(21) Appl. No.: 10/435,022

(22) Filed: May 12, 2003

Related U.S. Application Data
(60) Provisional application No. 60/388,368, filed on Jun. 14, 2002.

(30) Foreign Application Priority Data
May 10, 2002 (CA) 2,383,296

(51) Int. Cl. A61K 7/04; A61K 35/78; A61K 35/80

(52) U.S. Cl. 424/61; 424/195.17; 424/744; 424/748

(57) ABSTRACT

The present invention relates to a nail cosmetic composition comprising an extract of an alga and at least two components selected from the group consisting of an extract of a plant of the family of aloe vera, an extract of myrrh, keratin and a source of silica. The invention further relates to a nail cosmetic composition comprising mink oil and at least one vegetable oil. Methods for applying the compositions of the invention are also disclosed.

### TECHNICAL FIELD

The present invention relates to improvements in the field of nail care products. In particular, this invention relates to cosmetic compositions for improving the health of nails.

### BACKGROUND OF THE INVENTION

The nails of mammals, particularly the fingernails and toenails of humans, are composed of epidermis transparent cells called corneocyte cells, which are produced at the root of the nail. These cells join together to form a solid and continuous surface on the back of the terminal phalanges.

Each nail adheres intimately to the underlying support that forms the bed of the nail. The visible part of the nail is called the body of the nail. The hidden part is called the root.

The propagation of the corneocyte cells in the root ensures nail growth. The new cells go through a transformation in the solid layers and dry until they form a solid plate. The nail permanently receives continuous additions of cells at the level of its bed, which push the bed beyond the extremity of the phalange.

Keratin fibrils present inside the corneocyte cells, as well as the intercellular cements present, bring rigidity and flexibility to the nails.

Unfortunately, the fragility and vulnerability of the nails are observed in presence of mechanical, thermal and/or chemical attacks. For example, nails which are brittle and friable eventually break split or crack due to mechanical shocks. The nails may also be degraded in the presence of aggressive chemical agents, particularly those present in household products. Nail degradation may also be a secondary effect of the ageing process. Degradation of nails can be very painful, unpleasant and unsightly, and can permit foreign bodies to cause infection of the nails.

Numerous approaches have been investigated in order to improve the resistance of the nails to mechanical, thermal, chemical or temporal deterioration by hardening or strengthening them. Various compositions based on chemical products such as synthetic gums of an acrylic polymer film, varnishes based on nitrocellulose combined with inert glass fibers and other hardening agents have been proposed respectively in U.S. Pat. Nos. 5,508,027, 4,873,077, 4,482,538 and 5,785,959. Although these compositions may have an hardening or strengthening action on the nails, they do not allow one to improve the health of the nails. If any one of these compositions is applied to fragile or soft nails, harder nails may be obtained, but the latter will still have a tendency to crack and break.

Several approaches have also been investigated in order to improve the flexibility and resistance of the nails by nourishing and moisturizing them. Many attempts have been made to prevent the nails from dehydrating and to protect them against various types of deterioration. Compositions comprising synthetic active agents such as moisturizing, antimicrobial or antifungal agents have been extensively proposed.

U.S. Pat. No. 6,517,863 discloses compositions and methods for improving durability and hardness of the nail body. The compositions increase the durability of the nails by forming a protective layer of hydroxyapatite or other calcium phosphate mineral on the nail. Such compositions include synthetic and inorganic bloactive glasses as active agents. These inorganic glass materials are bound to the nails and comprise a plurality of synthetic chemicals products.

U.S. Pat. No. 5,645,823 discloses an aqueous solution for restoring and maintaining the compositional balance of normal, healthy keratinous tissues and their adjacent structures. The latter composition comprises a plurality of synthetic compounds used as antimicrobial or chelating agents or surfactants.

U.S. Pat. No. 4,919,920 discloses compositions and methods for reinforcing and hardening of mammals' keratinized appendages, wherein fluoride ions in an aqueous cosmetic carrier are topically applied. The utilization of fluoride ions increases both the hardness and the resistance of the nail. However, this method presents the drawback of generating in situ acidity. U.S. Pat. No. 5,478,551 also discloses the use of fluoride ions for strengthening nails. A non-aqueous organic composition containing ammonium hexafluorophosphate is used to provide an effective amount of fluoride ions to the nail body. In the latter two cases, harmful acidic by-products can potentially be formed.

Although the above-mentioned compositions may prevent brittleness of the nails or improve the health of nails, they all comprise synthetic chemicals as main active agents, in respect of which long term and/or secondary effects are still not completely known.

During the last decade, in the field of pharmaceutical products, nutrition and cosmetic care, the attitude of society with respect to synthetic products has changed considerably. The trend has been to avoid synthetic products as much as possible and replace them by natural or organic products. The emergence of functional food, neutraceuticals and natural cosmetic products represents a good example of this trend. People are now more aware of the fact that frequent or long term consumption or utilization of certain chemicals may have negative side effects on human health. Since many of these chemicals are quite recent, the long term effects are often unknown. Thus, natural or organic products have been extensively used as active agents in pharmaceutical or cosmetic products. For example, U.S. Pat. No. 5,667,768 discloses nail care compositions comprising an organic solution or dispersion of at least one natural active agent. However, the use of such compositions is tedious since they must be applied 2 to 3 times per week for at least 8 weeks in order to provide encouraging results.

### SUMMARY OF THE INVENTION

It is therefore an object of the present invention to overcome the above-mentioned drawbacks and to provide compositions for improving the health of nails, which comprise as main active ingredient a natural active agent.

According to one aspect of the invention, there is provided a nail cosmetic composition comprising an extract of an alga and at least two components selected from the group consisting of an extract of a plant of the family of aloe vera, an extract of myrrh, keratin and a source of silica.

According to another aspect of the invention, there Is provided a nail cosmetic composition comprising an extract of an alga selected from algae of the family Ulvaceae, together with a cosmetically acceptable carrier therefor.

According to still another aspect of the invention, there is provided a nail cosmetic composition comprising mink oil and at least one vegetable oil.

According to yet another aspect of the invention, there is provided a nail cosmetic composition comprising an extract of an alga and mink oil.

According to a further aspect of the invention, there is provided an active agent for improving the health of nails, comprising an extract of an alga selected from algae of the family Ulvaceae.

According to still a further aspect of the invention, there is provided an active agent for improving the health of nails, comprising mink oil.

According to yet a further aspect of the invention, there is provided a method of treating nails, comprising the step of applying to the nails a nail cosmetic composition according to the invention.

According to another aspect of the invention, there is provided a method of treating nails, comprising the steps of:

a) applying to the nails a first composition comprising an extract of an alga and at least one component selected from the group consisting of an extract of a plant of the family of aloe vera, an extract of myrrh, keratin and a source of silica; and

b) applying to the nails a second composition comprising mink oil and a cosmetically acceptable carrier.

Applicant has found quite surprisingly that by applying to the nails a nail cosmetic composition as defined above, the treated nails have improved health. The treated nails are more flexible, better-nourished and adequately moisturized. Moreover, the treated nails have a revitalized and healthy appearance and their growth rate is increased. Applicant has also found that the compositions of the invention can be used to treat the tissues surrounding the nail.

Even if the compositions of the invention can potentially contain minor proportions of a synthetic product, the main active agents used, such as an extract of an alga, mink oil, a vegetable oil, an extract of a plant of the family of aloe vera, an extract of myrrh, keratin or a source of silica, are natural and mild, and the compositions are thus not detrimental to the user's health.

The expression “a plant of the family of aloe vera” as used herein refers to plants of the Liliaceae family or lily family.

### DESCRIPTION OF PREFERRED EMBODIMENTS

In the compositions of the invention, the extract of an alga can be present in an amount of at least 5% by weight, based on the total weight of the composition. Preferably, the amount of the extract of an alga is comprised between 7 and 75%, and more preferably between 10 and 50% by weight.

In the active agents or the compositions of the invention, the alga can be selected from algae of the family Ulvaceae, and more particularly from algae of the genus Ulva. The alga is preferably selected from the group consisting of Ulva angusta, Ulva californica, Ulva costata, Ulva dactylifera, Ulva expansa, Ulva lobata, Ulva rigida, Ulva stenophyll, Ulva taeniata and Ulva lactuca. The alga is preferably Ulva lactuca, which is also known as sea lettuce. The extract of the alga can be a liquid extract.

The compositions of the invention can further comprise at least one component selected from the group consisting of an extract of a plant of the family of aloe vera, an extract of myrrh, keratin and a source of silica. The myrrh extracts used in the invention can also includes myrtle extract.

In the compositions of the invention, the extract of a plant of the family of aloe vera, the extract of a myrrh, the keratin or the source of silica can be present in an amount of 2 to 65% by weight, preferably 5 to 50% by weight, based on the total weight of the composition. The extract of a plant of the family of aloe vera is preferably a liquid extract. The keratin is preferably hydrolyzed keratin. The source of silica is preferably a horsetail extract. The horsetail extract can be selected from the genus Equisetum and is preferably Equisetum arvense.

Mink oil can be present in the compositions of the invention, in an amount of 3 to 85% by weight, based on the total weight of the composition. Preferably, the amount of mink oil is between 5 and 75%, and more preferably between 10 and 60% by weight. The mink oil is preferably emulsified into a cosmetically acceptable carrier.

The vegetable oil in the compositions of the invention can be almond oil, avocado oil, coconut oil, hazelnut oil, hemp oil, jojoba oil, linseed oil, olive oil, sesame oil, soya oil, pine oil or mixtures thereof. Preferably, the vegetable oil is selected from the group consisting of avocado oil, hemp oil and a mixture thereof. When use is made of a composition containing an extract of an alga and mink oil, the composition can further comprise a vegetable oil. The vegetable oil can be present in an amount of 5 to 85% by weight, based on the total weight of the composition. Preferably, the amount of vegetable oil is between 10 and 65% by weight. The vegetable oil is preferably emulsified into a cosmetically acceptable carrier.

The compositions of the invention can further comprise an additive selected from the group consisting of antibacterial agents, antifungal agents, antistatic agents, botanical extracts, fragrances, moisturizers, preservatives, UV filters, proteins, stabilizers, thickening agents, vitamins and mixtures thereof. The preservative is preferably DMDM hydantoin. The vitamin can be selected from the group consisting of DL-panthenol, phytantriol, vitamin E acetate and mixtures thereof. The thickening agent can be selected from the group consisting of fumed silica, hydrated silica, carrageenan, arrowroot and mixtures thereof. The thickening agent is preferably carrageenan. The compositions of the invention are preferably in liquid form.

The cosmetically acceptable carrier in the compositions of the invention can be present in an amount of at least 10% by weight, based on the total weight of the composition. Preferably, the amount of cosmetically acceptable carrier is at least 25% by weight. When use is made of a composition which includes an extract of an alga, the composition can further comprise a cosmetically acceptable carrier. The cosmetically acceptable carrier can be selected from the group consisting of a cream, a foam, a gel, a solution and a spray. Preferably, the cosmetically acceptable carrier is an aqueous solution or water. The solution can comprise 5 to 95% by weight of water, based on the total weight of the composition.

The compositions of the invention can be used for preventing demoisturization of nails, preventing friability of nails, improving the health of nails, providing an increased growth rate of nails and/or increasing the flexibility of nails.

In the methods of the present invention, the composition can be applied to the nails at least once a day for a period of at least 7 days and preferably of at least 14 days. The compositions are preferably capable of nourishing, flexibilizing, embellishing, revitalizing and/or moisturizing the nails.

When two compositions are applied to nails, the alga present in the first composition can be selected from algae of the family Ulvaceae, and more particularly from algae of the genus Ulva. The alga is preferably selected from the group consisting of Ulva angusta, Ulva californica, Ulva costata, Ulva dactylifera, Ulva expansa, Ulva lobata, Ulva rigida, Ulva stenophyll, Ulva taenlata and Ulva lactuca. In a particularly preferred embodiment, the alga is Ulva lactuca. The source of silica is preferably a horsetail extract. The cosmetically acceptable carrier present in the second composition is preferably a vegetable oil. The vegetable oil can be selected from the group consisting of almond oil, avocado oil, coconut oil, hazelnut oil, hemp oil, jojoba oil, linseed oil, olive oil, sesame oil, soya oil, pine oil and mixtures thereof. Preferably, the vegetable oil is selected from the group consisting of avocado oil, hemp oil and a mixture thereof. The first composition is advantageously allowed to penetrate into the nails prior to applying the second composition. Preferably, the nails are massaged after applying the first and second compositions so as to promote penetration of the compositions into the nails. Steps (a) and (b) can be performed at least once a day for a period of at least 7 days, preferably at least 14 days. The combination of the first and second compositions is advantageously capable of nourishing, flexibilizing, embellishing, revitalizing and/or moisturizing the nails. In an alternative method, step (b) can be performed prior to step (a).

The invention also relates to a nail cosmetic composition kit comprising a first composition and a second composition. The first composition can be any one of the compositions of the invention containing an extract of an alga and the second composition contains mink oil and cosmetically acceptable carrier, which is preferably a vegetable oil. The vegetable oil is preferably selected from the group consisting of avocado oil, hemp oil and a mixture thereof. The kit can further include at least one applicator for applying the first and second compositions to the nails. The kit can also comprise an instruction set effective for using the kit.

The nail cosmetic compositions of the present invention can be prepared by simply mixing together the cosmetic ingredients described hereinabove with stirring. The cosmetic ingredients are added one by one to form the desired composition and, after each addition, the mixture is stirred so as to be homogenized. Examples of satisfactory equipment and how to use it are readily apparent to one of ordinary skill in the art of nail care products.

The present invention will be more readily understood with reference to the following non-limiting examples.

### EXAMPLE 1

| Cosmetic ingredients | Wt % |
|---|---|
| DL-panthenol | 7.1 |
| hydroxy propyl trimonium | 20.6 |
| amino acids | |
| hydrolyzed keratin | 20.6 |
| horsetail extract | 10.3 |
| aloe extract | 10.3 |
| myrrh extract | 10.3 |
| Ulva lactuca extract | 20.6 |
| DMDM hydantoin | 0.2 |
| TOTAL | 100 |

### EXAMPLE 2

| Cosmetic ingredients | Wt % |
|---|---|
| mink oil | 30 |
| hemp oil | 30 |
| avocado oil | 30 |
| vitamin E acetate | 5 |
| phytantriol | 5 |
| TOTAL | 100 |

### EXAMPLE 3

| Cosmetic ingredients | Wt % |
|---|---|
| DL-panthenol | 4 |
| ethyl-panthenol | 10 |
| glycine | 2 |
| hydrolyzed silk | 2 |
| hydrolyzed keratin | 14 |
| hydroxy propyl trimonium | 4 |
| horsetail extract | 3 |
| aloe extract | 3 |
| myrrh extract | 3 |
| ulva lactuca extract | 3 |
| DMDM hydantoin | 0.4 |
| water | 51.6 |
| TOTAL | 100 |

The above-mentioned nail cosmetic compositions were tested so as to determine their efficacy for improving the health of nails. In order to perform such a test, two female test subjects were selected. The nails of test subjects 1 and 2 were treated with the nail cosmetic compositions of Examples 1 and 2.

Test Subject 1

Test subject 1 had extremely thin and brittle nails prior to treatment with the nail cosmetic composition of Example 1. In particular, the nails needed to be filed with a 600 grit file. At the beginning of the treatment, the subject's nails were cleansed using a nail polish remover and then, two layers of the nail cosmetic composition of Example 1 were applied to the nails. The nails were massaged in order to allow the composition to penetrate the nails and surrounding tissues. Then one layer of the nail cosmetic composition of Example 2 was applied to the nails and the nails were massaged. Such treatment was repeated by test subject 1 every day at bedtime. After one week of treatment, substantial improvement of the nails was noticed. In fact, the nails were revitalized, better nourished, more flexible and less breakable. Moreover, the nails and surrounding tissues were healthier looking. During the next three weeks, the treatment was maintained and continual improvement of the nails was noticed.

Test Subject 2

Test subject 2 had poor quality nails which were easily breakable and quite uneven. The nails needed to be filed with a 400 grit file. The same procedure as described for test subject 1 was applied to the nails of test subject 2. After one week of treatment, significant improvement was observed. The nails and surrounding tissues were healthier looking. The nails were revitalized, better nourished, more flexible and less breakable. During the next three weeks, the treatment was maintained and continual improvement of the nails was noticed. In particular, the nails were all even and the growth rate of the nails was increased. The growth rate of the nails was about 1/16 inch per week at the beginning of the treatment and increased to about 2/16 to 3/16 inch per week after the four-week treatment.

In view of the above results for test subjects 1 and 2, it is apparent that the nail cosmetic compositions of Examples 1 and 2 improve the health of the nails.

It has also been demonstrated that by using such nail cosmetic compositions, the improvement of the health of the nails is rapidly observed after one week of treatment. If the nail cosmetic of composition of Example 1 or 2 is used alone, encouraging results are observed in the nails of a test subject but the period or duration of the treatment will be longer. By using the nail cosmetic composition of Example 1 or 2 alone, two or three weeks may be required prior to observing substantial improvement of the health of the nails. In the above-mentioned treatment applied to the nails of test subjects 1 and 2, the nail cosmetic composition of Example 1 can alternatively be replaced with the nail cosmetic composition of Example 3. However, in such a case, the improvement of the health of the nails may be less rapid since the composition of Example 3 is less concentrated in active agents (horsetail extract, aloe extract, myrrh extract and alga extract) than the composition of Example 1.

It should be understood from the experiments carried out on the above-mentioned test subjects, that a treatment using the nail cosmetic compositions of the invention can be advantageously continued for more than four weeks and that by doing so, a user will still improve the health of the nails. The nail cosmetic compositions of the invention, if applied on a daily basis, permit the user to have adequately nourished nails and surrounding tissues which are in good health. The nail cosmetic compositions of the Invention can be applied to the nails by a professional nail technician or by the customer.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A nail cosmetic composition comprising an extract of an alga and at least two components selected from the group consisting of an extract of a plant of the family of aloe vera, an extract of myrrh, keratin and a source of silica.

2. The composition of claim 1, wherein said extract of an alga is present in an amount of at least 5% by weight, based on the total weight of the composition.

3. The composition of claim 2, wherein the amount of said extract of an alga is between 10 and 50% by weight.

4. The composition of claim 1, wherein said alga is selected from algae of the family Ulvaceae.

5. The composition of claim 4, wherein said alga is selected from algae of the genus Ulva.

6. The composition of claim 5, wherein said alga is Ulva Lactuca.

7. The composition of claim 1, wherein said extract of a plant of the family of aloe vera is present in an amount of 5 to 50% by weight, based on the total weight of the composition.

8. The composition of claim 1, wherein said extract of a plant of the family of aloe vera is a liquid extract.

9. The composition of claim 1, wherein said extract of a myrrh is present in an amount of 5 to 50% by weight, based on the total weight of the composition.

10. The composition of claim 1, wherein said keratin is present in an amount of 5 to 50% by weight, based on the total weight of the composition.

11. The composition of claim 1, wherein said source of silica is present in an amount of 5 to 50% by weight, based on the total weight of the composition.

12. The composition of claim 11, wherein said source of silica is a horsetail extract.

13. The composition of claim 1, further including a cosmetically acceptable carrier.

14. A nail cosmetic composition comprising an extract of an alga selected from algae of the family Ulvaceae, together with a cosmetically acceptable carrier therefor.

15. The composition of claim 14, wherein said alga is selected from algae of the genus Ulva.

16. The composition of claim 15, wherein said alga is Ulva Lactuca.

17. The composition of claim 14, wherein said extract is present in an amount of at least 5% by weight, based on the total weight of the composition.

18. The composition of claim 17, wherein the amount of said extract is between 10 and 50% by weight.

19. The composition of claim 14, wherein said cosmetically acceptable carrier is present in an amount of at least 25% by weight, based on the total weight of the composition.

20. The composition of claim 14, wherein said cosmetically acceptable carrier is selected from the group consisting of a cream, a foam, a gel, a solution or a spray.

21. The composition of claim 20, wherein said cosmetically acceptable carrier is an aqueous solution.

22. The composition of claim 14, further including at least one component selected from the group consisting of an extract of a plant of the family of aloe vera, an extract of myrrh, keratin and a source of silica.

23. A nail cosmetic composition comprising mink oil and at least one vegetable oil.

24. The composition of claim 23, wherein said mink oil is present in an amount of 5 to 75% by weight, based on the total weight of the composition.

25. The composition of claim 24, wherein the amount of said mink oil is between 10 and 60% by weight, based on the total weight of the composition.

26. The composition of claim 23, wherein said vegetable oil is selected from the group consisting of almond oil, avocado oil, coconut oil, hazelnut oil, hemp oil, jojoba oil, linseed oil, olive oil, sesame oil, soya oil, pine oil and mixtures thereof.

27. The composition of claim 23, wherein said vegetable oil is present in an amount of 5 to 85% by weight, based on the total weight of the composition.

28. The composition of claim 27, wherein the amount of said vegetable oil is between 10 and 65% by weight based on the total weight of the composition.

29. A nail cosmetic composition comprising an extract of an alga and mink oil.

30. The composition of claim 29, further including a cosmetically acceptable carrier.

31. The composition of claim 30, wherein said mink oil is emulsified into said cosmetically acceptable carrier.

32. The composition of claim 29, further including at least one component selected from the group consisting of a vegetable oil, an extract of a plant of the family of aloe vera, an extract of myrrh, keratin and a source of silica.

33. The composition of claim 32, wherein said vegetable oil is emulsified into said cosmetically acceptable carrier.

34. An active agent for improving the health of nails, comprising an extract of an alga selected from algae of the family Ulvaceae.

35. The active agent of claim 34, wherein said alga is selected from algae of the genus Ulva.

36. The active agent of claim 35, wherein said alga is Ulva Lactuca.

37. An active agent for improving the health of nails, comprising mink oil.

38. A method of treating nails, comprising the step of applying to the nails a nail cosmetic composition comprising an extract of an alga and at least two components selected from the group consisting of an extract of a plant of the family of aloe vera, an extract of myrrh, keratin and a source of silica.

39. The method of claim 38, wherein said composition is applied to the nails at least once a day for at least 7 days.

40. The method of claim 39, wherein said composition is applied to the nails at least once a day for a period of at least 14 days.

41. The method of claim 38, wherein said composition is capable of nourishing, flexibilizing, embellishing, revitalizing or moisturizing the nails.

42. A method of treating nails, comprising the steps of:
a) applying to the nails a first composition comprising an extract of an alga and at least one component selected from the group consisting of an extract of a plant of the family of aloe vera, an extract of myrrh, keratin and a source of silica; and
b) applying to the nails a second composition comprising mink oil and a cosmetically acceptable carrier.

43. The method of claim 42, wherein the nails are massaged after applying said first and said second compositions for promoting penetration of said compositions into the nails.

44. The method of claim 42, wherein steps (a) and (b) are performed at least once a day for a period of at least 7 days.

45. The method of claim 42, wherein the combination of said first and second compositions is capable of nourishing, flexibilizing, embellishing, revitalizing or moisturizing the nails.

46. A nail cosmetic composition kit including a first composition comprising an extract of an alga and at least two components selected from the group consisting of an extract of a plant of the family of aloe vera, an extract of myrrh, keratin and a source of silica, and a second composition comprising mink oil and a cosmetically acceptable carrier.

47. The kit of claim 46, further including at least one applicator for applying said first and said second compositions to the nails or a set of instructions effective for using said kit.
